(19) United States
(12) Patent Application Publication
(10) Pub. No.: US 2011/0085982 A1
(43) Pub. Date: Apr. 14, 2011

(54) DKAT CELL LINE, A MODEL FOR HUMAN TRIPLE-NEGATIVE BREAST CANCER

(75) Inventors: Victoria L. Seewaldt, Durham, NC (US); Nicholas C. D'Amato, Durham, NC (US); Julie H. Ostrander, Golden Valley, NC (US)

(73) Assignee: Duke University; The Regents of the University of California

(21) Appl. No.: 12/901,292

(22) Filed: Oct. 8, 2010

Related U.S. Application Data
(60) Provisional application No. 61/250,083, filed on Oct. 9, 2009.
(60) Provisional application No. 61/250,164, filed on Oct. 9, 2009.

(51) Int. Cl. A61K 49/00; A01K 67/027; C12N 5/00; C12N 5/071; C12Q 1/68; C12Q 1/02

(52) U.S. Cl. 424/9.2; 800/10; 435/325; 435/366; 435/6; 435/29

(57) ABSTRACT

The present invention provides a human triple-negative breast cancer cell line designated DKAT. The DKAT cell line has a marker profile of high expression of Snail-1 and Snail-2 (Slug); and a p53 mutation in exon 8 at codon 273 (CGT>CAT). The present invention further provides methods of using the DKAT cell line.

### STATEMENT OF PRIORITY

This application claims the benefit, under 35 U.S.C. §119 (e), of U.S. Provisional Application No. 61/250,083, filed on Oct. 9, 2009, and U.S. Provisional Application No. 61/250,164, filed on Oct. 9, 2009, the entire contents of each of which are incorporated by reference herein.

### STATEMENT OF GOVERNMENT SUPPORT

Aspects of this invention were supported by funding provided under National Institute of Health/National Cancer Institute Grant No. 5RO1-CA098441, 2R01CA088799, P30CA14236, and 2R01CA114068. The U.S. Government has certain rights to this invention.

### FIELD OF THE INVENTION

The present invention provides a human triple negative carcinoma cell line and methods of making and using the cell line.

### BACKGROUND OF THE INVENTION

Breast cancer is a heterogeneous disease, which exhibits a wide range of clinical behaviors, prognoses, and histologies (Tavassoli F, Devilee P, editors. (2003) WHO Classification of Tumors. Pathology & Genetics: Tumors of the breast and female genital organs. Lyon (France): IARC Pres). Gene expression profiling has recently identified specific breast cancer subgroups, each with a distinct molecular signature (2-4). This molecular classification system divides breast cancers into four biologically different subtypes: 1) normal/non-cancerous (expression profile similar to noncancerous breast tissue); 2) estrogen-receptor (ER)-positive luminal breast cancer [expression of luminal cytokeratins (CK 8, 18, and 19) and E-cadherin]; 3) ER-negative, HER2/neu overexpressing breast cancer [overexpression of ERBB2 oncogene]; and 4) ER-negative triple-negative breast cancer [expression of basal-type cytokeratins (CK 5/6, 14, 17) p63, and overexpression of epidermal growth factor receptor (EGFR)] (2-4).

Triple-negative breast cancers are typically observed in young African American women and Caucasian women who carry a mutation in the BRCA1 gene (2-4). Triple-negative breast cancers are typically ER/PR(−/−), Her2/neu(−/−), EGFR(+), p53(−) and cytokeratin 5/6 (+I+). While some triple-negative breast cancers respond to chemotherapy, a subset of triple-negative breast cancers are chemotherapy-resistant and highly metastatic, carrying with it an extremely poor prognosis (2-7). The molecular mechanism which governs the aggressive behavior of this subset of triple-negative breast cancers is a matter of intense speculation, particularly since triple-negative breast cancers frequently express markers of epithelial mesenchymal transition (EMT).

EMT is a normal developmental process in which cells of epithelial origin lose epithelial characteristics and polarity, and acquire a mesenchymal phenotype associated with increased migratory behavior (8-14). EMT is characterized by loss of expression of epithelial makers (CK 8/18); increased expression of mesenchymal markers (vimentin and smooth muscle actin); loss of intercellular adhesion (E-cadherin and occludins); acquisition of a spindle-like morphology, cytoskeleton reorganization; and increased motility, invasiveness, and metastasic capabilities (10-14). In addition, EMT is associated with “cadherin switching” (down-regulation of E-cadherin and up-regulation of the mesenchymal cadherins, N-cadherin or cadherin-11) and the accumulation of h-catenin (12, 14, 15, 16). Gene expression changes of specific transcription factors are associated with EMT, including Snail/Snail1 (17), Slug/Snail2 (18), SIP-1/ZEB-2 (19), yEF1/ZEB-1 (20), E12/E47 (21), and Twist (22). These factors act as transcriptional repressors of E-cadherin (23, 24) and are thought to modulate directly or indirectly the expression of genes involved in cancer invasion and metastasis, and thereby promote EMT in vitro (25, 26).

It is hypothesized that EMT and mesenchymal-epithelial transition (MET) may occur during invasion and metastasis of breast cancer and that breast cancers that have the capacity to undergo EMT/MET may have an increased malignant potential (10-13). In support of this hypothesis is the recent observation that EMT markers are frequently expressed in triple-negative breast cancer (27). However, controversy exists regarding the precise definition of EMT as well as the existence of EMT/MET during human carcinogenesis (28-30). Much of the evidence for the association of EMT/MET with breast cancer invasion is derived from studies in animal models (Damonte et al. Breast Cancer Res. 2008 10:R50 (doi:10.1186/bcr2104) and Debies et al. J. Clin. Invest. J. Clin. Invest. 118(1):51-63 (2008)) and breast cancer cell lines that are highly adapted to tissue culture conditions (12, 13). It is often difficult to identify EMT/MET in human breast cancer because the full sequence of events defining EMT/MET in vitro is not commonly observed in vivo (29). Therefore, the relevance of EMT to human breast cancer is a matter of intense debate (8, 12, 13, 29). Some investigators have proposed that EMT may be transient and reversible, and may only occur in isolated foci confined to the invasive segment of breast cancers (12, 31).

Studies of breast cancers have provided evidence of self-renewing, stem-like cells, which have been called cancer initiating cells (CICs). CICs constitute a small minority of neoplastic cells within a tumor and are defined operationally by their ability to seed new tumors. For this reason, they have also been termed “tumor-initiating cells” (32). For example, a small subpopulation of cancer cells is present within some human breast cancers that exhibit a CD44high/CD24low antigenic phenotype. These cells are highly enriched for tumor-initiating cells in comparison to the majority of carcinoma cells of the CD44low/CD24high phenotype found in the same tumors (33). Triple-negative breast cancers have been reported to have a higher percentage of CD44high/CD24low than other breast cancer subtypes (34). It has been observed that stem-like cells isolated either from mouse or human mammary glands or breast cancer express markers of EMT, illustrating a potential link between the capacity of breast cancer cells to undergo EMT/MET and the gain of epithelial stem cell properties (35).

Breast cancer cell lines which accurately model the various subtypes of the disease are an invaluable tool for the study of breast cancer and the development of novel therapeutics. Unfortunately, there have historically been few breast cancer cell lines which accurately model the properties of human triple-negative breast cancer. The present invention overcomes previous shortcomings in the art by providing a cell line that is valuable as a model of triple-negative breast cancer.

### SUMMARY OF THE INVENTION

The present invention provides a novel model of triple-negative breast cancer which is a cell-line that is tumorigenic in mice and comprises a putative “breast cancer stem cell” population. This cell line has a marker profile of high expression of Snail-1 and Snail-2 (Slug); and a p53 mutation in exon 8 at codon 273 (CGT>CAT) and is deposited under ATCC Accession No. PTA-10322.

Accordingly, the present invention provides a cell line deposited under ATCC Accession No. PTA-10322.

Additionally provided herein is a human triple-negative breast cancer cell line, wherein the cell line comprises the following marker profile: high expression of Snail-1 and Snail-2 (Slug); and a p53 mutation in exon 8 at codon 273 (CGT>CAT).

In some embodiments of the present invention a human triple-negative breast cancer cell line is provided, wherein the cell line comprises the following marker profile: high expression of Snail-1 and Snail-2 (Slug); and a p53 mutation in exon 8 at codon 273 (CGT>CAT) and is deposited under ATCC Accession No. PTA-10322.

The present invention further provides a human triple-negative breast carcinoma cell line, wherein said cell line produces a solid carcinoma upon subcutaneous implantation or injection into a non-human mammal, said cell line comprising the following marker profile: high expression of Snail-1 and Snail-2 (Slug); and a p53 mutation in exon 8 at codon 273 (CGT>CAT).

Furthermore, the present invention provides a solid tumor produced in a non-human mammal, wherein said tumor is produced by introducing into said mammal a cell or cells of a human triple-negative breast cancer cell line, wherein said cell line comprises the following marker profile: high expression of Snail-1 and Snail-2 (Slug); and a p53 mutation in exon 8 at codon 273 (CGT>CAT).

Also provided is a non-human mammal comprising one or more cells of a human triple-negative breast cancer cell line, wherein the cell line comprises the following marker profile: high expression of Snail-1 and Snail-2 (Slug); and a p53 mutation in exon 8 at codon 273 (CGT>CAT).

Another aspect of the present invention is a method of producing a solid tumor in a non-human mammal, comprising introducing into said non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell line produces a solid tumor in said mammal.

In other embodiments, a method is provided for establishing a cell line derived from a human triple-negative breast cancer tumor, comprising: a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; b) removing said tumor from said non-human mammal; and c) culturing the cells of the tumor in a culture medium, thereby establishing a cell line derived from a human triple-negative breast cancer tumor.

The present invention additionally provides a method of identifying modulation of gene and/or protein expression and/or activity during breast cancer invasion, comprising: a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; and b) identifying modulation of gene and/or protein expression and/or activity during breast cancer invasion resulting from the presence of said solid tumor.

A further aspect of the present invention is a method of identifying modulation of gene expression during metastasis, comprising: a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; and b) identifying modulation in gene expression during metastasis resulting from the presence of said solid tumor.

A still further aspect of the present invention provides a method of identifying modulation in gene expression during epithelial to mesenchymal transition and mesenchymal to epithelial transition, comprising: a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; and b) identifying modulation in gene expression during epithelial to mesenchymal transition and mesenchymal to epithelial transition resulting from the presence of said solid tumor.

The present invention also provides a method of identifying a chemotherapeutic agent having a therapeutic effect on a triple-negative breast cancer cell, comprising: a) contacting a human triple-negative breast cancer cell comprising the following marker profile: high expression of Snail-1 and Snail-2 (Slug); and a p53 mutation in exon 8 at codon 273 (CGT>CAT) with a chemotherapeutic agent; and b) determining if the chemotherapeutic agent inhibits proliferation of the triple-negative breast cancer cell, whereby identification of a chemotherapeutic agent that inhibits proliferation the triple-negative breast cancer cell identifies a chemotherapeutic agent having a therapeutic effect on a triple-negative breast cancer cell.

In some aspects of the present invention, a method is provided of identifying a chemotherapeutic agent having a therapeutic effect on triple-negative breast cancer, comprising: a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; b) administering a chemotherapeutic agent to the non-human mammal with said solid tumor; and c) determining if the chemotherapeutic agent inhibits proliferation of the tumor, whereby identification of a chemotherapeutic agent that inhibits tumor proliferation identifies a chemotherapeutic agent having a therapeutic effect on triple-negative breast cancer.

In further embodiments, the present invention provides a method of identifying a biological agent having a therapeutic effect on a triple-negative breast cancer cell, comprising: a) contacting a human triple-negative breast cancer cell comprising the following marker profile: high expression of Snail-1 and Snail-2 (Slug); and a p53 mutation in exon 8 at codon 273 (CGT>CAT) with a biological agent; and b) determining if the biological agent inhibits the proliferation of the triple-negative breast cancer cell, whereby identification of a biological agent that inhibits proliferation of the triple-negative breast cancer cell identifies a biological agent having a therapeutic effect on a triple-negative breast cancer cell.

In still further embodiments, the present invention provides a method of identifying a biological agent having a therapeutic effect on triple-negative breast cancer, comprising: a) introducing a cell of a human triple-negative breast cancer cell line comprising the following marker profile: high expression of Snail-1 and Snail-2 (Slug); and a p53 mutation in exon 8 at codon 273 (CGT>CAT) into a non-human mammal, wherein said cell produces a solid tumor in said non-human mammal; b) administering a biological agent to the non-human mammal with said solid tumor; and c) determining if the biological agent inhibits proliferation of the solid tumor, whereby identification of a biological agent that inhibits tumor proliferation identifies a biological agent having a therapeutic effect on triple-negative breast cancer.

The present invention also provides a method of screening for modulators of gene expression, comprising: a) measuring the level of gene expression in a cell from a cell line of this invention; b) contacting said cell with a candidate agent; c) measuring the level of gene expression in said cell after contact with the candidate agent, whereby a difference in level of gene expression after contact with the candidate agent as compared to before contact with the candidate agent identifies a modulator of gene expression.

These and other aspects of the invention will be set forth in more detail in the description of the invention that follows.

### BRIEF DESCRIPTION OF THE FIGURES

FIG. 1 shows spectral karyotypic analyses of the DKAT cell line.

FIGS. 2A-F show central nervous system (CNS) metastasis to the brain parenchyma (FIGS. 2A-C) and to the leptomenges (FIG. 2D and FIG. 2E) in immunocompromised mice injected in carotid artery with DKAT cells. FIG. 2F shows that these same mice also developed lung metastasis.

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

Triple-negative breast cancer is characterized as being estrogen receptor (ER)-negative, progesterone receptor (PR)-negative and lacking amplification of the HER2/neu gene. Triple-negative breast cancer typically affects young African American women and Caucasian women who carry a mutation of the BRCA1 gene. While some triple-negative breast cancers respond to chemotherapy, many are highly resistant to all treatments. There is a great need to develop new therapies for treatment-resistant triple-negative breast cancer. However, progress in developing therapies for treatment-resistant triple-negative breast cancer is currently limited due to a lack of adequate models of treatment resistant-triple-negative breast cancer. While the breast cancer cell line MDA-MB-231 has been proposed to be a model of triple-negative breast cancer, differential gene expression studies characterize MDA-MB-213 as “mesenchymal” (in other words, not epithelial, and therefore not representative of triple-negative breast cancers, which are epithelial in origin). Other common cell lines currently used as a triple-negative breast cancer model include SUM-149 and MCF10A cell lines. Although classified as triple-negative, the SUM-149 line is actually derived from an inflammatory breast cancer (IBC) and the MCF10A line does not form tumors in mouse models.

The present invention provides a novel model of chemotherapy-resistant triple-negative breast cancer in which tumors form in mice and which is useful for the study of triple-negative mammary carcinogenesis and the development of novel therapeutics targeting this lethal subtype of breast cancer.

### DEFINITIONS

As used herein, “a,” “an” or “the” can mean one or more than one. For example, a cell can mean a single cell or a multiplicity of cells.

As used herein, “and/or” refers to and encompasses any and all possible combinations of one or more of the associated listed items, as well as the lack of combinations when interpreted in the alternative (or).

Further, the term “about,” as used herein when referring to a measurable value such as an amount of a compound or agent, dose, time, temperature, and the like, is meant to encompass variations of ±20%, ±10%, ±5%, ±1%, ±0.5%, or even±0.1% of the specified amount.

As used herein, nucleic acids encompass both RNA and DNA, including cDNA, genomic DNA, mRNA, synthetic (e.g., chemically synthesized) DNA and chimeras of RNA and DNA. The nucleic acid can be double-stranded or single-stranded.

The term “isolated” can refer to a nucleic acid or polypeptide that is substantially free of cellular material, viral material, or culture medium (e.g., when produced by recombinant DNA techniques), or chemical precursors or other chemicals (e.g., when chemically synthesized). Moreover, an isolated fragment is a fragment of a nucleic acid or polypeptide that is not naturally occurring as a fragment and would not be found in the natural state. Furthermore, an isolated cell is a cell that has been separated from other components with which it is normally associated in nature. For example, an isolated cell can be a cell in culture medium.

More specifically, an isolated nucleic acid is a DNA or RNA that is not immediately contiguous with nucleotide sequences with which it is immediately contiguous (one on the 5′ end and one on the 3′ end) in the naturally occurring genome of the organism from which it is derived. In other embodiments, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic nucleic acid of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid encoding an additional polypeptide or peptide sequence.

The term “cell line,” as used herein, encompasses individual cells, harvested cells, and/or cultures containing the cells, so long as they are derived from cells of the cell line referred to. A cell line is said to be “continuous,” “immortal,” or “stable” if the line remains viable over a prolonged time, typically at least about six months. To be considered a cell line, as used herein, the cells are present in a culture environment and must remain viable for at least 40 passages. A human cell line of the present invention is comprised of cells that have human chromosomes and can be maintained continuously in cell culture conditions for over 70 passages.

A cell line is said to be “tumorigenic” if, when the cell line is injected into a host animal, the host animal develops a tumor or cancer that is anaplastic, invasive, and/or metastatic. A “human” tumor is comprised of cells that have human chromosomes. Such tumors include those in a human patient, and tumors resulting from the introduction of a cell or cells from a human malignant cell line into a non-human host animal if cells from such tumors have human chromosomes.

The terms “modulate,” “modulates,” modulated” or “modulation” refer to enhancement (e.g., an increase) or inhibition (e.g., a reduction) in the specified activity (e.g., modulated protein production). A modulator is an agent (e.g., a chemical agent; a biological agent, or a chemotherapeutic agent, and the like) which modulates an activity (e.g., gene and/or protein expression and/or activity, and the like).

The terms “anticancer agent,” “conventional anticancer agent,” or “cancer therapeutic drug” refer to any therapeutic agents (e.g., chemotherapeutic agents or compounds and/or molecular therapeutic compounds), radiation therapies, or surgical interventions, used in the treatment of cancer (e.g., in mammals).

As used herein, the term “chemotherapeutic agent” refers to a pharmacologic agent that is used in the treatment of cancer. It is intended that the term “chemotherapeutic agent” encompass anti-hyperproliferative and antineoplastic agents as well as other biologically therapeutic compounds. The term “anti-neoplastic agent” refers to any compound that retards, slows, and/or inhibits the proliferation, growth, and/or spread of a targeted (e.g., malignant) neoplasm. Thus, these agents have the ability to inhibit cancer cell proliferation (e.g., tumor proliferation) and/or kill cancer cells. Common chemotherapeutic agents are well known to those skilled in the art.

A chemotherapeutic agent of this invention can be, but is not limited to, irinotecan, gemcytobine, cisplatin, carboplatin, procarbazine, mechlorethamine, cyclophosphamide, camptothecin, ifosfamide, melphalan, chlorambucil, busulfran, nitrosurea, dactinomycin, daunorubicin, doxorubicin, bleomycin, plicamycin, mitomycin, etoposide, tamoxifen, raloxifene, estrogen receptor binding agents, taxol, paclitaxol, gemcitabine, navelbine, farnesyl-protein transferase inhibitors, transplatinum, 5-fluorouracil, floxuridine, mutamycin, vincristin, vinblastin, methotrexate, MEK kinase inhibitors, antibodies, small molecules (e.g., HERCEPTIN monoclonal antibody, tyrosine kinase inhibitors, signal transduction inhibitors, etc.), as well as any analogue or derivative of a chemotherapeutic agent of this invention.

Further chemotherapeutic agents include but are not limited to mitomycin C, Vitamin K3, 2,5-diaziridinyl-3-(hydroxymethyl)-6-methyl-1,4-benzoquinone (i.e., RHI), 2,5-dimethyl-3,6-diaziridinyl-1,4-benzoquinone (i.e., MeDZQ), and beta-lapachone. A chemotherapeutic agent of this invention can be present in a composition of this invention and/or employed in a method of this invention in any combination with other chemotherapeutic agents and/or other therapeutic agents. Dosage ranges for the chemotherapeutics of this invention would be known and/or readily determined by one skilled in the art.

As used herein “biological agent” refers to a substance that is made from a living organism or its products and is used in the prevention, diagnosis, and/or treatment of cancer and other diseases. Biological agents include antibodies, antibiotics, anti-virals, interleukins, agents that alter protein phosphorylation and/or protein activity, block or inhibit receptor function, alter DNA methylation, effect DNA repair, alter protein expression, alter RNA expression, alter RNA splicing, and/or vaccines.

The term “test compound” or “candidate agent” refers to any chemical entity, pharmaceutical, drug, and the like, that can be used to treat and/or prevent a disease, illness, sickness, or disorder of bodily function, and/or otherwise alter the physiological and/or cellular status of a sample (e.g., the level of Bcl-2 family proteins in a cell). Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment and/or prevention.

An “effective” amount as used herein is an amount of a compound or composition hat is sufficient to achieve the intended effect, e.g., to treat and/or prevent a disorder in a subject. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science and Practice of Pharmacy (20th ed. 2000)).

When used in a therapeutic context, an “effective” amount is an amount sufficient to provide some improvement or benefit to the subject, e.g., an amount that provides some alleviation, mitigation or decrease in at least one clinical symptom, a delay or reduction in the progression of the disorder, and/or prevention or delay of the onset of the disorder. Those skilled in the art will appreciate that the therapeutic effects need not be complete or curative, as long as some benefit is provided to the subject.

By the term “treat,” “treating” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder. The terms “treat,” “treats,” “treating,” or “treatment of” and the like also include prophylactic treatment of the subject. As used herein, the terms “prevent,” “prevents,” or “prevention” (and grammatical equivalents thereof) are not meant to imply complete abolition of disease and encompass any type of prophylactic action that reduces the incidence of the condition, delays the onset and/or progression of the condition, and/or reduces the symptoms associated with the condition. Thus, unless the context indicates otherwise, the terms “treat,” “treating” or “treatment of” (or grammatically equivalent terms) refer to both prophylactic and therapeutic regimens.

The present invention provides a cell line deposited under ATCC Accession No. PTA-10322. The cell line of the present invention, deposited under ATCC Accession No. PTA-10322, is further designated as the “DKAT cell line.” The DKAT cell line is characterized by the following markers as set forth below in Table 1. Identification of these markers was carried out according to methods well known in the art for identifying markers in cells as well as according to methods described in the Examples herein (see, e.g., Perou et al. Nature 406, 747-752 (2000); Kreike et al. Breast Cancer Res. 9:R65 (2007); Nielsen et al. Clinical Cancer Res. 10, 5367-74 (2004); Emad et al. Breast Cancer Res, 9:204 (2007); Korkaya et al. PLoS Biology 7 (6):e1000121 (June 2009)).

TABLE 1
Epithelial to mesenchymal markers

| | |
|---|---|
| Epithelial markers: | Cytokeratin 8/18-negative |
| | Cytokeratin 19-negative |
| | E-cadherin-low expression, |
| | primarily cytoplasmic |
| Mesenchymal markers: | Vimentin-high |
| | Smooth muscle actin-high |
| | Cytokeratin 5/6-high |
| | Cytokeratin 14-high |
| | Cytokeratin 17 |
| | Snail-1-high |
| | Snail-2(Slug)-high |
| Triple-negative breast cancer markers | Cytokeratin 8/18-negative |
| | Cytokeratin 5/6-positive |
| | ER/PR negative |
| | Her2/neu not amplified |
| | EGFR overexpressing |
| | p53 mutant (mutation exon 8 |
| | at codon 273 (CGT > CAT)) |
| | p63 high |
| | Twist positive |
| Stem cell markers | CD44^{+high}/CD24^{-/low} |
| | Aldehyde dehydrogenase positive |
| | Notch- positive |
| Other | AKT-pSer473 high |

The p53 mutant refers to a mutation in tumor protein p53 (TP53). The TP53 gene is located on the short (p) arm of chromosome 17 at position 13.1. Mutations in the TP53 gene are well known in the art to be associated with various types of cancer including breast cancer. The mutation associated with triple-negative breast cancer is found in exon 8 at codon 273 ((CGT>CAT).

Accordingly, in some embodiments, an isolated human triple-negative breast cancer cell line (DKAT) is provided, wherein the cell line comprises the following marker profile: high expression of Snail-1 and Snail-2 (Slug); and a p53 mutation in exon 8 at codon 273 (CGT>CAT).

The present invention further provides a human triple-negative breast carcinoma cell line, wherein said cell line produces a solid carcinoma (i.e., tumor) upon subcutaneous implantation and/or injection into a non-human mammal, said cell line comprising the following marker profile: high expression of Snail-1 and Snail-2 (Slug); and a p53 mutation in exon 8 at codon 273 (CGT>CAT). In some embodiments, the human triple-negative breast carcinoma cell line comprising the marker profile of high expression of Snail-1 and Snail-2 (Slug); and a p53 mutation in exon 8 at codon 273 (CGT>CAT) is deposited under ATCC Accession No. PTA-10322.

Methods of subcutaneous or mammary implantation of a cell into an animal are known in the art. (See e.g., Mani et al. Cell 133, 704-715, 2008; Abbey, et al. PNAS 101:11438-43, 2004; U.S. Pat. No. 6,949,690)

A non-human animal of the present invention includes any non-human mammal susceptible to breast cancer. Such a non-human mammal includes, but is not limited to, primate, dog, cat, pig, mouse, rabbit, guinea pig, goat, bovine, horse, and the like. Thus, in some embodiments, a non-human animal can be any domestic, commercially and/or clinically valuable animal. In particular embodiments, the non-human animal of the present invention is a mouse.

In some embodiments, the non-human mammal can be immune deficient. In further embodiments, the non-human animal comprises T-cell immunosuppression. In yet further embodiments, the non-human animal is an immune deficient mouse. In still further embodiments, the non-human animal is a mouse having T-cell immunosuppression.

Immunosuppressed or immune deficient non-human animals of the present invention include severe combined immune deficient (SCID) mice. Various other immune deficient mice, rodents or animals may be used, including those which are deficient as a result of a genetic defect, which may be naturally occurring or induced, such as, for example, nude mice (a strain of mice with a genetic mutation (FOXN1) gene that causes a deteriorated or absent thymus gland, resulting in an inhibited immune system due to a greatly reduced number of T cells), Rag 1 and/or Rag 2 mice, and the like, and mice that have been cross-bred with these mice and have an immunocompromised background. The deficiency may be, for example, as a result of a genetic defect in recombination, a genetically defective thymus and/or a defective T-cell receptor region. Induced immune deficiency may be as a result of administration of an immunosuppressant, e.g., cyclosporin, removal of the thymus, etc. Various transgenic immune deficient mice are currently available and/or can be developed in accordance with conventional techniques. Ideally, the immune deficient mouse will have a defect that inhibits maturation of lymphocytes, particularly lacking the ability to rearrange the T-cell receptor region. In addition to mice, immune deficient rats or similar rodents may also be employed in the practice of this invention. (See e.g., U.S. Pat. No. 6,949,690).

Thus, in some embodiments, the cell line of the present invention produces a solid carcinoma (e.g., tumor) upon subcutaneous implantation and/or injection into a mouse. In other embodiments, the cell line of the present invention produces a solid carcinoma upon subcutaneous implantation or injection into an immune deficient mouse having T-cell immunosuppression.

The present invention additionally provides a solid tumor produced in a non-human mammal wherein said tumor is produced by introducing into said mammal a cell or cells of a human triple-negative breast cancer cell line, wherein said cell line comprises the following marker profile: high expression of Snail-1 and Snail-2 (Slug); and a p53 mutation in exon 8 at codon 273 (CGT>CAT). In particular embodiments, the present invention provides a solid tumor produced in a non-human mammal wherein said tumor is produced by introducing into said mammal a cell or cells of a human triple-negative breast cancer cell line, wherein said cell line comprises the cell line deposited under ATCC Accession No. PTA-10322.

Also provided is a non-human mammal comprising one or more cells of a human triple-negative breast cancer cell line, wherein the cell line comprises the following marker profile: high expression of Snail-1 and Snail-2 (Slug); and a p53 mutation in exon 8 at codon 273 (CGT>CAT). In particular embodiments, the present invention provides a non-human mammal comprising one or more cells of a human triple-negative breast cancer cell line, wherein said cell line comprises the marker profile of high expression of Snail-1 and Snail-2 (Slug); and a p53 mutation in exon 8 at codon 273 (CGT>CAT), and is deposited under ATCC Accession No. PTA-10322.

In some embodiments, the invention provides an immune deficient mouse comprising a cell or cells of the cell line having the marker profile of high expression of Snail-1 and Snail-2 (Slug); and a p53 mutation in exon 8 at codon 273 (CGT>CAT).

The present invention also provides a method of producing a solid tumor in a non-human mammal comprising introducing into said non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said mammal. In particular embodiments of the invention, a method of producing a solid tumor in a non-human mammal is provided comprising introducing into said non-human mammal a cell or cells of a human triple-negative breast cancer cell line, wherein said cell line comprises the marker profile of high expression of Snail-1 and Snail-2 (Slug); and a p53 mutation in exon 8 at codon 273 (CGT>CAT), and is deposited under ATCC Accession No. PTA-10322, further wherein said cell or cells produce a solid tumor in said mammal.

In some embodiments, the invention provides a method of producing a solid tumor in an immune deficient mouse, comprising introducing into said mouse a cell of a cell line having the marker profile of high expression of Snail-1 and Snail-2 (Slug); and a p53 mutation in exon 8 at codon 273 (CGT>CAT), wherein said cell produces a solid tumor in said mouse.

In other embodiments, the present invention provides a method for establishing a cell line derived from a human triple-negative breast cancer tumor, comprising a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; b) removing said tumor from said non-human mammal; and c) culturing a cell of said tumor in a culture medium, thereby establishing a cell line derived from the human triple-negative breast cancer tumor.

In still other embodiments, the present invention provides a method for establishing a cell line derived from a human triple-negative breast cancer tumor, comprising: a) introducing into a non-human mammal a cell of a human triple-negative breast cancer cell line; wherein said cell line comprises the marker profile of high expression of Snail-1 and Snail-2 (Slug); and a p53 mutation in exon 8 at codon 273 (CGT>CAT) and is deposited under ATCC Accession No. PTA-10322, further wherein said cell produces a solid tumor in said non-human mammal; b) removing said tumor from said non-human mammal; and c) culturing a cell of said tumor in a suitable culture medium, thereby establishing a cell line derived from the human triple-negative breast cancer tumor.

In some embodiments, the invention provides a method for establishing a cell line derived from a human triple-negative breast cancer tumor in an immune deficient mouse, comprising introducing into said mouse a cell of a human triple-negative breast cancer cell line having the marker profile of high expression of Snail-1 and Snail-2 (Slug); and a p53 mutation in exon 8 at codon 273 (CGT>CAT), wherein said cell produces a solid tumor in said mouse, removing said tumor from said mouse, and then culturing cells of said tumor in a culture medium, thereby establishing a cell line derived from a human triple-negative breast cancer tumor.

The DKAT cell line of the present invention is useful for studying the pathogenesis and treatment of triple-negative breast cancer. For example, immune deficient mice bearing subcutaneous (and/or other) xenografts may be used to evaluate the effect of various treatments for triple-negative breast cancer (e.g., therapeutic compositions, gene therapies, immunotherapies, etc.) on the growth of tumors and progression of disease. Xenograft cells may be used to identify novel genes and/or genes that are differentially expressed in triple-negative breast cancer cells, and/or to analyze the effect such genes have on the progression of triple-negative breast cancer. In addition, triple-negative breast cancer xenograft cells may be used for the introduction of various genetic capabilities, including the introduction of various genes, antisense sequences, ribozymes, regulatory sequences which enhance or repress the expression of endogenous genes, and so forth.

Accordingly, provided herein is a method of identifying modulation of gene and/or protein expression and/or activity during breast cancer invasion, comprising: a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; and b) identifying modulation of gene and/or protein expression and/or activity during breast cancer invasion resulting from the presence of said tumor.

In particular embodiments, a method of identifying modulation of gene and/or protein expression and/or activity during breast cancer invasion is provided, comprising: a) introducing into a non-human mammal a cell of a human triple-negative breast cancer cell line, wherein said cell line comprises the following marker profile: high expression of Snail-1 and Snail-2 (Slug); and a p53 mutation in exon 8 at codon 273 (CGT>CAT) and is deposited under ATCC Accession No. PTA-10322, wherein said cell produces a solid tumor in said non-human mammal; and b) identifying modulation of gene and/or protein expression and/or activity during breast cancer invasion resulting from the presence of said tumor.

In some embodiments, the invention provides a method of identifying modulation of gene and/or protein expression and/or activity during breast cancer invasion, comprising introducing into a mouse a cell of a cell line having the marker profile of high expression of Snail-1 and Snail-2 (Slug); and a p53 mutation in exon 8 at codon 273 (CGT>CAT), wherein said cell produces a solid tumor in said mouse and identifying modulation of gene and/or protein expression and/or activity during breast cancer invasion resulting from the presence of said tumor.

In further embodiments, a method of identifying modulation of gene and/or protein expression and/or activity during breast cancer invasion is provided, comprising introducing into a non-human mammal a cell of a human triple-negative breast cancer cell line, wherein said cell line comprises the following marker profile: high expression of Snail-1 and Snail-2 (Slug); and a p53 mutation in exon 8 at codon 273 (CGT>CAT) and is deposited under ATCC Accession No. PTA-10322, and observing modulation of gene and/or protein expression and/or activity during breast cancer invasion resulting from introduction of said cell.

In some embodiments of the invention, the modulation of gene and/or protein expression and/or activity includes, but is not limited to, modulation (e.g., increase or decrease) of gene copy number, miRNA level(s) and type, mRNA level(s), protein level(s), protein phosphorylation, methylation, promoter analysis, protein binding, and any combination thereof. Assays for measuring these activities are well known in the art (Mani et al. Cell 133:704-715 (2008)).

Also provided herein is a method of identifying modulation of gene expression during metastasis, comprising: a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; and b) identifying modulation in gene expression during metastasis resulting from the presence of said solid tumor.

In particular, provided herein is a method of identfying modulation of gene expression during metastasis, comprising: a) introducing into a non-human mammal a cell of a human triple-negative breast cancer cell line, wherein said cell line comprises the marker profile of high expression of Snail-1 and Snail-2 (Slug); and a p53 mutation in exon 8 at codon 273 (CGT>CAT), and is deposited under ATCC Accession No. PTA-10322, further wherein said cell of said cell line produces a solid tumor in said non-human mammal; and b) identifying modulation in gene expression in said mammal during metastasis resulting from the presence of said solid tumor.

In some embodiments, the invention provides a method of identifying modulation of gene expression during metastasis, comprising: introducing into a mouse a cell of a human triple-negative breast cancer cell line having the marker profile of high expression of Snail-1 and Snail-2 (Slug); and a p53 mutation in exon 8 at codon 273 (CGT>CAT), wherein said cell of said cell line produces a solid tumor in said mouse, and identifying modulation of gene expression in said mouse during metastasis resulting from the presence of said solid tumor.

In further embodiments, the invention provides a method of identifying modulation of gene expression during metastasis, comprising: introducing into a non-human mammal a cell or cells of a human triple-negative breast cancer cell line having the marker profile of high expression of Snail-1 and Snail-2 (Slug); and a p53 mutation in exon 8 at codon 273 (CGT>CAT); and identifying modulation of gene expression in said non-human mammal during metastasis resulting from introduction of said cell.

The present invention additionally provides a method of identifying modulation in gene expression during epithelial to mesenchymal transition and mesenchymal to epithelial transition, comprising: a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; and b) identifying modulation in gene expression during epithelial to mesenchymal transition and mesenchymal to epithelial transition resulting from the presence of said solid tumor.

In particular, the present invention provides a method of identifying modulation in gene expression during epithelial to mesenchymal transition and mesenchymal to epithelial transition, comprising: a) introducing into a non-human mammal a cell of a human triple-negative breast cancer cell line, wherein said cell line comprises the following marker profile: high expression of Snail-1 and Snail-2 (Slug); and a p53 mutation in exon 8 at codon 273 (CGT>CAT), and is deposited under ATCC Accession No. PTA-10322, further wherein said cell produces a solid tumor in said non-human mammal; and b) identifying modulation in gene expression during epithelial to mesenchymal transition and mesenchymal to epithelial transition in said mammal resulting from the presence of said solid tumor.

In some embodiments, the invention provides a method of identifying modulation of gene expression during epithelial to mesenchymal transition and mesenchymal to epithelial transition, comprising: introducing into a mouse a cell of a human triple-negative breast cancer cell line having the marker profile of high expression of Snail-1 and Snail-2 (Slug); and a p53 mutation in exon 8 at codon 273 (CGT>CAT), wherein said cell produces a solid tumor in said mouse; and identifying modulation of gene expression in said mouse during epithelial to mesenchymal transition and mesenchymal to epithelial transition resulting from the presence of said solid tumor.

In further embodiments, the invention provides a method of identifying modulation of gene expression during epithelial to mesenchymal transition and mesenchymal to epithelial transition, comprising introducing into a non-human mammal a cell of a human triple-negative breast cancer cell line having the marker profile of high expression of Snail-1 and Snail-2 (Slug); and a p53 mutation in exon 8 at codon 273 (CGT>CAT); and identifying modulation of gene expression in said non-human mammal during epithelial to mesenchymal transition and mesenchymal to epithelial transition resulting from introduction of said cell or cells.

Genes useful for identifying modulation of gene and/or protein expression during breast cancer invasion, during metastasis and during epithelial to mesenchymal transition and mesenchymal to epithelial transition as disclosed in the present invention include but, are not limited to, the genes provided in Table 2 herein.

TABLE 2
Epithelial to mesenchymal markers

| Epithelial markers: | Cytokeratin 8/18 |
|---|---|
| | Cytokeratin 19 |
| | E-cadherin |
| | Occludin |
| Mesenchymal markers: | Vimentin |
| | Smooth muscle actin |
| | Cadherin 11 |
| | N-cadherin |
| | Cytokeratin 5/6 |
| | Cytokeratin 14 |
| | Cytokeratin 17 |
| | Snail-1 |
| | Snail-2(Slug) |
| | SIP-1/ZEB-2 |
| | yEF1/ZEB-1 |
| | E12/E47 |
| | Twist |
| | TGF-beta |
| Triple-negative breast cancer markers | Cytokeratin 8/18-negative |
| | Cytokeratin 5/6-positive |
| | ER/PR negative |
| | Her2/neu not amplified |
| | EGFR overexpressing |
| | p53 mutant (mutation exon 8 |
| | at codon 273 (CGT > CAT)) |
| | p63 high |
| Stem cell markers | CD44⁺/CD24^{-/low} |
| | Aldehyde dehydrogenase |
| | Wnt |
| | p21 |
| | Notch |
| Invasion | MMP-9 |
| | MMP-2 |
| | MMP-7 |
| | HIF-1alpha |
| | Rho-GTPases |
| | PAI-1 |
| | IGF-1 |
| | Wnt |
| | EpCAM |
| | Ras |
| | TGFalpha |
| | N-cadherin |
| | VEGF |
| | NF-kappaB |
| | Smad |
| | TGF-beta |
| | Snail-1 |
| | Snail-2/Slug |
| | AKT |
| | 14-3-3 |

Thus, non-limiting examples of genes and/or proteins for which the gene and/or protein expression assays can be carried out include transcription factors (e.g., snail, slug), AKT, c-Src, paxillin, FAK, epithelial growth factor (EGFR) signaling proteins, PTEN signaling proteins, p53 signaling proteins, cell cycle regulatory proteins (e.g. p21, p63, pRB, cyclin dependent kinase proteins), AKT-signaling proteins, transforming growth receptor-beta (TGF-beta) signaling proteins, structural proteins (e.g., integrins, laminin, claudins, cell contact proteins), apoptosis regulatory proteins, mitochondrial signaling proteins, steroid receptors, co-activators, and any combination thereof.

Various art-known assays are available for determining protein and/or gene expression. These assays include, but are not limited to, differential nRNA transcript expression (Perou et al. Nature 406:747-752 (2000)); methylation analysis; methylation-specific PCR, which is a bisulfite conversion based PCR technique for the study of DNA CpG methylation (Derks et al. Cellular Oncology, 26:291-299 (2004)); high throughput methylation profiling including Sequenom® MassARRAY® system (Sequenom, Inc., San Diego, Calif.), which utilizes MALDI-TOF mass spectrometry in combination with RNA base specific cleavage (MassCLEAVE™ kit) (Sequenom, Inc., San Diego, Calif.); single nucleotide polymorphism analysis; MethyLight™ analysis, a bisulfite modification-dependent fluorescence-based real time PCR assay (Eads et al. Nucleic Acids Res. 28(8) e32 (2000); Erhlich et al., Oncogene 21:6694-6702 (2002)); imprinting analysis using reverse transcription-polymerase chain reaction (Nakao et al. Hum. Mol. Genet. 3:309-315 (1994)); microsatellite analysis (Modrich, P., J. Biol. Chem. 281:30305-30309 (2006); chromatin immunoprecipitation assay (ChIP) (Mulero-Navarro et al., Carcinogenesis 27:1099-1104 (2006); Nakagawachi et al., Oncogene 22:8835-8844 (2003)), mammosphere culture (Mani et al. Cell 133:704-715 (2008)), Western analysis, pyrosequencing (Lee et al. Clinical Cancer Research 14:2664-2672 (2008); Dejeux et al., J. Mol. Diagn. 9:510-520 (2007); Lavebratt et al. Nat. Protoc. 1(6):2573-82 (2006)); and the like as are well known in the art.

Detection of methylation of the CpG islands according to methods of this invention includes but is not limited to methylation specific-polymerase chain reaction (MS-PCR), a method of nucleic acid amplification that is well known in the art. In this assay, bisulfite modification of the DNA sequence allows the detection of differences between methylated and unmethylated alleles. Reaction of the DNA with sodium bisulfite converts all unmethylated cytosines to uracil, which is recognized as thymine by Taq polymerase, but does not affect methylated cytosines. Amplification with primers specific for methylated or unmethylated DNA discriminates between methylated and unmethylated DNA. This assay provides a simple and fast way of surveying multiple samples to detect methylation of cytosines in the region of interest (Widschwendter et al., “Methylation and silencing of the retinoic acid receptor-beta2 gene in breast cancer” J. Natl. Cancer Inst. 92(10):826-832 (2000)).

The present invention also provides assays for determining the effect of candidate agents or candidate therapeutic compositions or treatments on the growth of triple-negative breast cancer cells. In one embodiment, the assay comprises applying the agent, composition and/or treatment to a SCID or other immune deficient mouse bearing a subcutaneous human triple-negative breast cancer xenograft and determining the effect of the treatment on the growth of the xenograft.

Thus, the present invention provides a method of identifying a chemotherapeutic agent having a therapeutic effect on a triple-negative breast cancer cell, comprising: a) contacting a human triple-negative breast cancer cell comprising the following marker profile: high expression of Snail-1 and Snail-2 (Slug); and a p53 mutation in exon 8 at codon 273 (CGT>CAT) with a chemotherapeutic agent; and b) determining if the chemotherapeutic agent inhibits proliferation of the triple-negative breast cancer cell, whereby identification of a chemotherapeutic agent that inhibits proliferation of a triple-negative breast cancer cell identifies a chemotherapeutic agent having a therapeutic effect on a triple-negative breast cancer cell. In some particular embodiments, said cell line is the DKAT cell line deposited under ATCC Accession No. PTA-10322.

Also provided herein is method of identifying a chemotherapeutic agent having a therapeutic effect on triple-negative breast cancer, said method comprising: a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; b) administering a chemotherapeutic agent to said non-human mammal with said solid tumor; and c) determining if the chemotherapeutic agent inhibits proliferation of the tumor, whereby identification of a chemotherapeutic agent that inhibits tumor proliferation identifies a chemotherapeutic agent having a therapeutic effect on triple-negative breast cancer. In some embodiments, said human triple-negative breast cancer cell line is the DKAT cell line deposited under ATCC Accession No. PTA-10322.

In some embodiments, the invention provides a method of identifying a chemotherapeutic agent having a therapeutic effect on triple-negative breast cancer, comprising: a) introducing into said mouse a cell of a human triple-negative breast cancer cell line having the marker profile of high expression of Snail-1 and Snail-2 (Slug); and a p53 mutation in exon 8 at codon 273 (CGT>CAT), wherein said cell produces a solid tumor in said mouse; b) administering a chemotherapeutic agent to said mouse with said solid tumor; and c) determining if the chemotherapeutic agent inhibits proliferation of the tumor, whereby identification of a chemotherapeutic agent that inhibits tumor proliferation identifies a chemotherapeutic agent having a therapeutic effect on triple-negative breast cancer.

In further embodiments, the present invention provides a method of identifying a biological agent having a therapeutic effect on a triple-negative breast cancer cell, comprising: a) contacting a human triple-negative breast cancer cell comprising the following marker profile: high expression of Snail-1 and Snail-2 (Slug); and a p53 mutation in exon 8 at codon 273 (CGT>CAT) with a biological agent; and b) determining if the biological agent inhibits the proliferation of the triple-negative breast cancer cell, whereby identification of a biological agent that inhibits proliferation of the triple-negative breast cancer cell identifies a biological agent having a therapeutic effect on a triple-negative breast cancer cell. In some particular embodiments, said cell is of the human triple-negative breast cancer cell line, DKAT, deposited under ATCC Accession No. PTA-10322.

In still further embodiments, the present invention provides a method of identifying a biological agent having a therapeutic effect on triple-negative breast cancer, comprising: a) introducing into a non-human mammal a cell of a human triple-negative breast cancer cell line comprising the following marker profile: high expression of Snail-1 and Snail-2 (Slug); and a p53 mutation in exon 8 at codon 273 (CGT>CAT), wherein said cell produces a solid tumor in said non-human mammal; b) administering a biological agent to the non-human mammal with said solid tumor; and c) determining if the biological agent inhibits proliferation of the solid tumor, whereby identification of a biological agent that inhibits tumor proliferation identifies a biological agent having a therapeutic effect on triple-negative breast cancer. In some embodiments, said human triple-negative breast cancer cell line is the DKAT cell line deposited under ATCC Accession No. PTA-10322.

In some embodiments, the invention provides a method of identifying a biological agent having a therapeutic effect on triple-negative breast cancer comprising: a) introducing into said mouse a cell of a human triple-negative breast cancer cell line having the marker profile of high expression of Snail-1 and Snail-2 (Slug); and a p53 mutation in exon 8 at codon 273 (CGT>CAT); b) administering a biological agent to said mouse with said solid tumor; and c) determining if the biological agent inhibits proliferation of the tumor, whereby identification of a biological agent that inhibits tumor proliferation identifies a biological agent having a therapeutic effect on triple-negative breast cancer

The present invention also provides a method of screening for modulators of gene expression, comprising: a) measuring the level of gene expression in a cell from a cell line of the present invention; b) contacting said cell with a candidate agent; c) measuring the level of gene expression in said cell after contact with the candidate agent, whereby a difference in gene expression level after contact with the candidate agent as compared to before contact with the candidate agent identifies a modulator of gene expression. In some embodiments, said cell line of the present invention is from the DKAT cell line deposited under ATCC Accession No. PTA-10322. Thus, the measuring of the level of gene expression in part (a) provides the baseline (steady state) level of gene expression in the cell from the cell line of the present invention against which is compared the level of gene expression in the cell after contact with the candidate agent. The types of genes for which gene expression can be measure include, but are not limited to, those involved in invasion, metastasis, apoptosis, motility, epithelial to mesenchymal transition, cell cycle regulatory genes, and transcription factors.

The present invention will now be described with reference to the following examples. It should be appreciated that these examples are for the purpose of illustrating aspects of the present invention, and do not limit the scope of the invention as defined by the claims.

### EXAMPLES

### Example 1

Establishment of the DKAT Culture: The DKAT cell line was isolated from the pleural effusion of a 35 year old woman with basal-type breast cancer who initially presented with a 4 cm ER/PR(−/−), Her2/Neu (−/−), cytokeratin 5/6(+/+), EGFR(+) lymph node-negative breast cancer (T2NOMO). The patient was treated with taxane- and cytoxan-based chemotherapy and radiation, but the cancer rapidly progressed locally within the radiation field and also metastasized to the lung, liver, and bone.

Pleural fluid from the woman was obtained in accordance with Institutional Review Board guidelines of The Ohio State University. Cells from the pleural fluid were pelleted, resuspended grown, and maintained in mammary epithelial cell growth medium (MEGM) supplemented with 52 μg/ml bovine pituitary extract, 5 μg/ml insulin, 10 ng/ml human recombinant epidermal growth factor, 0.5 μg/ml hydrocortisone (Lonza, Basel, Switzerland) at 37° C. in a humidified incubator with 5% CO₂/95% air. DKAT cells are split 1:3 once a week, and have been kept in continuous culture for over 3 years (>70 passages). Mycoplasma testing was performed as previously reported (Seewaldt et al., J. Cell Biol. 155:471-86 (2001)).

### Example 2

Other Cell Lines Primary human mammary epithelial cells (HMECs) (Lonza, Basel, Switzerland) were immortalized with hTERT (HMEC-hTERT), and maintained in supplemented MEBM as above. MDA-MB-231 and MCF-7 cells (American Type Culture Collection, Manassas, Va.) were maintained in minimal essential medium alpha (MEMalpha), supplemented with 5% fetal bovine serum, 10 ng/ml epidermal growth factor, 5 μg/ml insulin, 0.5 μg/ml hydrocortisone.

### Example 3

Cytogenetic analysis: Spectral karyotypic analyses (SKY) was performed as previously described (Seewaldt et al., J. Cell Biol. 155:471-86 (2001); Mrozek et al., Genes Chromosomes Cancer 6:249-252 (1993)). Karyotypic abnormalities were classified according to the International System for Human Cytogenetic Nomenclature (F, Mitelman, ISCN: International System for Human Cytogenetic Nomenclature, Basel, Switzerland, Karger, S. (1995)).

### Example 4

Immunocytochemisty: The human formalin-fixed, paraffin-embedded primary breast biopsy, chest wall recurrence, and bone metastasis was sectioned at a 4 um thickness and stained for alpha-smooth muscle actin (1A4, Sigma), vimentin (3B4 Boehringer Mannheim Roche, Hvidovre, Denmark), keratin CK7 (OVTM, DAKO, Glostrup, Denmark) and wide range keratins (MNF116, DAKO). The antibodies were visualized by streptavidin-biotin (DAKO 5004).

### Example 5

Immunofluorescence: Cells were plated at 1×10⁵ cells/well in 4-well chamber slides (Thermo Fisher, Rochester, N.Y.) and incubated at 37° C., 5% CO₂ overnight. For cytokeratin staining, cells were washed twice with 1×PBS and then fixed with ice cold methanol for 20 minutes. Cells were washed twice with 1×PBS and then permeabilized for 10 minutes with 1×PBS containing 0.1% Triton X-100. Samples were blocked for 30 minutes in 1×PBS containing 0.05% Triton X-100, 5% BSA and 2% goat serum. Primary antibodies for cytokeratin 5, cytokeratin 8, cytokeratin 17, E-cadherin, EGFR, ERα, and anti-p63 are from Santa Cruz Biotechnology Inc. (Santa Cruz, Calif.), Vimetin (BD biosciences), N-cadherin and P-Cadherin are from Abeam Inc. (Cambridge, Mass.), and progesterone receptor is from Thermo Fisher Scientific Inc. (Fremont, Calif.). Primary antibodies were diluted in blocking solution and samples were incubated overnight at 4° C. Following three 1×PBS washes, samples were incubated in goat anti-mouse Alexa-fluor 488 and/or goat anti-rabbit Alexa-fluor 597 secondary antibodies from Invitrogen (Carlsbad, Calif.) for one hour. Samples were washed twice with 1×PBS and then incubated for 5 minutes in 1×PBS containing 5 ug/mL DAPI, and then washed two additional times. Slides were mounted with Vectashield and stored at 4° C. overnight prior to imaging. All images were taken on a Zeiss Axio Observer A1 fluorescence microscope (Carl Zeiss, Göttingen, Germany) with a 63× oil objective.

### Example 6

Western Blotting Preparation of cellular lysates and immunoblotting were performed as previously described (Seewaldt et al., J. Cell Biol. 155:471-86 (2001)). For p53 expression, the blocked membrane was incubated with a 1:1000 dilution of the p53 antibody (DO-1, Santa Cruz, Santa Cruz, Calif.). For Akt-pSer473 expression, the blocked membrane was incubated with a 1:200 dilution of the Akt-pSer473 antibody (4051) (Cell Signaling, Danvers, Mass.) and for Akt-1, Akt-2, Akt-3 isoforms expression, the blocked membrane was incubated with a 1:200 dilution of the antibodies, 2967, 2964, 3788, respectively (Cell Signaling, Danvers, Mass.)). Loading control was provided by 1:1000 dilution of the 119 antibody to beta-actin (Santa Cruz, Santa Cruz, Calif.). The resulting film images were digitized and quantitated using Kodak 1D Image Analysis Software (Eastman Kodak, Rochester, Minn.).

### Example 7

p53 sequencing: Sequencing of exons 5-9 of the TP53 gene using previously published primers and programs (IARC TP53 Mutation Database, World Health Organization; IARC Database Manager, Group of Molecular Carcinogenesis and Biomarkers, 150, cours Albert Thomas, 69372 LYON CEDEX 08[www-p53.iarc.fr/p53sequencing.html]) was performed on genomic DNA isolated from passage 20 DKAT cells. Mutations were tested in both the sense and antisense sequences, and were confirmed by repeat sequencing of a second, independent genomic DNA preparation.

### Example 8

Differential Gene Expression Studies: mRNA was collected using the Qiagen RNeasy mini kit with the optional DNAse step (Quiagen, Valencia, Calif.). RNA integrity was confirmed by electrophoresis, and samples were stored at −80° C. until used. All RNA combinations used for array analysis were obtained from cells harvested at identical confluency. cDNA synthesis and probe generation for cDNA array hybridization were obtained by following the standardized protocols provided by Affymetrix™ (Affymetrix, Santa Clara, Calif.). Expression data for approximately 5,600 full-length human genes was collected using Affymetrix GeneChip HuGeneFL™ arrays, following the standardized protocols provided by the manufacturer and as previously published (Dietze et al., J. Cell Sci. 118:5005-5022 (2005)). Array images were processed using Affymetrix MAS 5.0 software. Probe-level signals were filtered for saturation and intra-array probe intensity data was scaled to a target intensity of 1000. Data was collected in triplicate using independent biological replicates and p-values were calculated for pair-wise comparisons using CyberT Software (Baldi et al. Bioinformatics 17:509-519 (2001)).

Median expression values of technical replicates (n=3) for each of three cell lines, HMECs, DKAT, and MDA-MB-231, were compared with published breast cancer cell line expression data (Charafe-Jauffret et al., Oncogene 25(15):2273-84 (2006)). Probe values for the current study were background corrected, quantile normalized, and summarized with RMA (Irizarry et al., Nucleic Acids Res. 31(4):e15 (2003)) using the Affymetrics package implemented in BRB array tools (Simon and Lam, ver. 3.7.0; R ver. 2.6.0). All data were log₂ transformed. Probe Set IDs were used to merge the two data sets (16,383 features). Duplicate Gene Symbols were summarized by mean Probe Set ID values. The data were array- and gene-wise median centered and filtered to exclude genes with standard deviations of observed values less than 1.5. The remaining 473 features were clustered using centroid linkage and Pearson correlation as the similarity metric (Cluster ver. 3.0). The results were displayed using Java TreeView (ver. 1.1.0).

### Example 9

Invasion Assays: Transwell invasion assays were performed as previously described (Lochter et al. J. Biol. Chem., 272, 5007-5015 (1997). Briefly, 1×10⁵ DKAT (passages 3-15) or MDA-MB-231 cells were plated on the inner chamber of 8 μm pore size 24 well-size filters coated with growth factor-reduced matrigel (BD Biosciences, San Jose, Calif.) or uncoated control inserts. Cells were plated in base MEBM medium+0.01% FBS. Base MEBM+10% FBS was used as the chemoattractant. Plates were incubated at 37° C. for 20 hours. Assays with DKAT and MDA-MB-231 cells were performed in triplicate in two independent experiments. Numbers shown are the average of triplicate wells, three fields counted per well.

### Example 10

EMT Assay: To induce EMT, DKAT cells were grown in stromal cell growth media (SCGM) supplemented with 5 μg/ml insulin, 1 ng/ml FGF-2, and 5% fetal bovine serum (FBS) (Lonza, Basel, Switzerland).

### Example 11

Flow Cytometry of DKAT cells: Allophycocyanin (APC)-conjugated anti-CD44 and phycoerythrin (PE)-conjugated anti-CD24 antibodies were obtained from BD Biosciences (San Jose, Calif.). Briefly, cells were harvested from culture flasks using Cell Dissociation Buffer (Invitrogen, Carlsbad, Calif.), washed with PBS containing 1% BSA, and incubated with 7-aminoactinomycin D (7-AAD, Invitrgen, Carlsbad, Calif.), anti-CD44 and anti-CD24 antibodies for 30 minutes in the dark at room temperature. After washing again with PBS containing 1% BSA, cells were analyzed for CD44/CD24 expression. Fluorescence of analysis of DKAT cells was performed using a FACSCaliber flow cytometer and analyzed with CellQuest software (Becton Dickinson Immunocytometry Systems). Forward and side scatter were used to establish size gates and exclude cellular debris, and dead cells were eliminated from the analysis by gating 7-AAD-negative cells only. Twenty to thirty thousand events were collected.

### Example 12

Mouse xenograft: Cells were stained for CD24 and CD44 as described and sorted using the FACSVantage (BD Biosciences). Doublets and higher order clumps were excluded using a time-of-flight approach, where forward-scatter-height was plotted against forward scatter area. CD44⁺/CD24^{low} (lowest ⅓ of CD24 staining) and CD44⁺/CD24^{high} (highest ⅓ of CD24 staining) populations were isolated, resuspended in PBS, and matrigel (BD Biosciences, San Jose, Calif.) was added at a 1:1 ratio. For unsorted cell injections, cells were detached from tissue culture containers using cell dissociation buffer and live cells were counted using a trypan blue exclusion method. After rinsing with PBS, cells were resuspended in 1:1 PBS:Matrigel. A total of 50 μl of cells in the PBS/Matrigel mixture were injected into the uncleared #4 mammary fat pad of NOD.CB17-PrkdcSCID/J mice. The number of injected cells ranged from 1×10⁴ to 10 cells in 10-fold dilutions. Mice were monitored over time and tumor growth was assessed. Mice were sacrificed when the tumor reached 1.5 cm in diameter, or after 150 days. Tumors were fixed in 4% paraformaldehyde at 4° C. overnight.

### Example 13

DKAT line: The DKAT cell line was isolated from the pleural effusion of a 35 year old Caucasian woman with no family history of breast or ovarian cancer, who initially presented with a 4 cm ER/PR(−/−), Her2/Neu (−/−), cytokeratin 5/6(+/+), EGFR(+) lymph node-negative triple-negative breast cancer (T2N0M0). The woman underwent mastectomy, followed by chemotherapy, and radiation therapy. The primary breast cancer was resistant to cyclophosphamide, methotrexate, and 5-fluorouracil chemotherapy, recurred within the radiation field, and rapidly metastasized to the lung, pleura, liver, and bone. The woman developed pancytopenia; bone marrow biopsy demonstrated extensive marrow replacement with tumor cells. The tumor progressed through taxotere and navelbine and pleural fluid was obtained. Two weeks later the patient expired from rapid progression of disease.

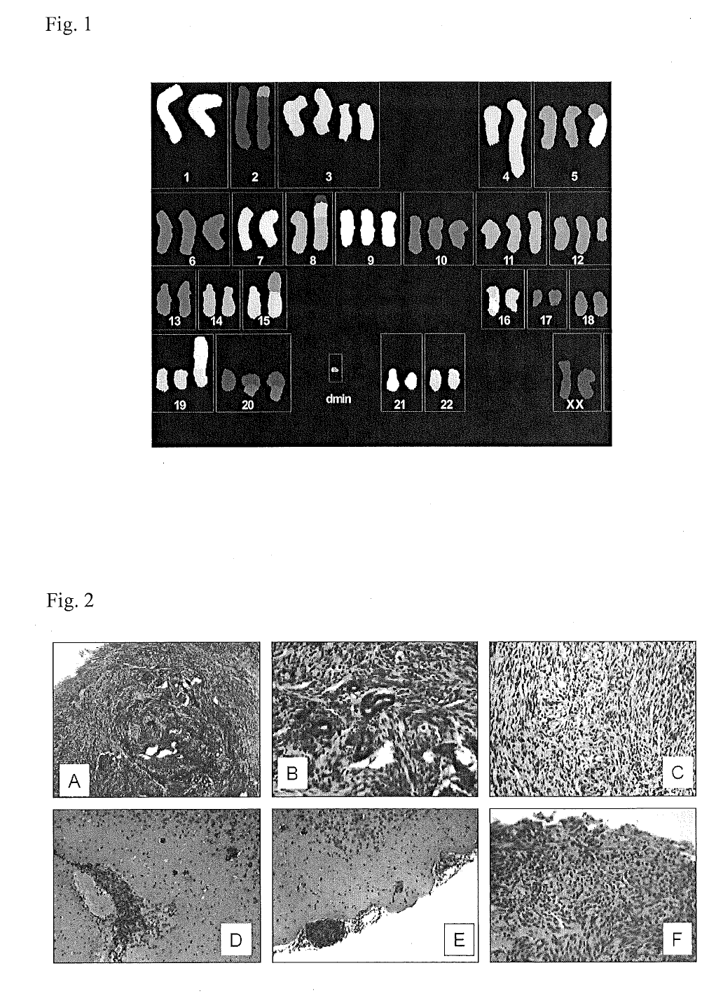

DKAT cells isolated from the pleural effusion of the patient grew immediately in culture, did not require an adaptation period, and retained the basal phenotype. Karyotyping of the DKAT line was performed at passage 3-5. SKY karyotyping confirmed a near diploid karyotype of 49XX, with translocations involving 17q11.1, 17p, and deletion of chromosome 3p12 (FIG. 1). A stable population of DKAT cells exhibits high expression of CD44 and low expression of CD24, the phenotype of the putative “breast cancer stem cell.” The DKAT cell line has been maintained continuously in culture for over 3 years (>70 passages). Doubling time is 24 hours.

Cytogenetic Analysis and Fluorescence in situ Hybridization: Twenty-five metaphase cells of the DKAT cell line (passage 3) were subjected to spectral karyotyping (SKY) analysis and an additional 9 cells were G-banded and karyotyped (FIG. 1). The modal chromosome number was 56. In addition to 29 cells with the hyperdiploid chromosome number, 3 cells had hypopentaploid chromosome number (104-109 chromosomes) and 2 cells had, respectively, 117 and 127 chromosomes. Among the predominant hyperdiploid cells, two clones, a stemline and a sideline 1, were identified. The only difference between the stemline and sideline 1 is the presence of one double minute (dmin) in a sideline. Because of its small size, the origin of this double minute could not be identified with confidence. Sideline 2 is near tetraploid and represents a doubling of the stemline, with some random chromosome losses and a few non-clonal aberrations in the cells, and sideline 3 is a composite karyotype of 2 cells with the highest chromosome numbers.
DKAT cells express triple-negative markers and markers of EMT: Expression of markers of triple-negative breast cancer and of immunohistochemical markers of EMT was analyzed in the primary tumor, bone metastasis, and DKAT cell line. Triple-negative markers included ER/PR, HER2, EGFR, p63, CK 5/6, CK 14, CK 17, CK 8/18, and p53; and markers associated with EMT included vimentin, E-, N-, P-cadherin, and snail (Table 3).

TABLE 3
Characterization of Primary Tumor, Metastasis, and DKAT cells.

| | | Chest Wall | Bone Marrow | | |
|---|---|---|---|---|---|
| Marker | Primary | Recurrence | Metastasis | DKAT line | Basal |
| ER | −(OSU) | | | — | — |
| PR | −(OSU) | | | — | — |
| HER2 | Not amplified | | | Not amplified | Not amplified |
| | (OSU) | | | | |
| EGFR | | | | Overexpressed | Overexpressed |
| CK5/6 | ++(UCD) | ++(UCD) | ++(UCD) | ++ | ++ |
| CK14 | +(UCD) | +(UCD) | +(UCD) | ++ | ++ |
| CK17 | | | | ++ | ++ |
| CK8 | (+)(UCD) | (+)(UCD) | (+)(UCD) | (+) | — |
| CK18 | (+)(UCD) | (+)(UCD) | (+)(UCD) | (+) | — |
| E-cadherin | +*(UCD) | +*(UCD) | +*(UCD) | +(cytoplasmic)? | — |
| P-cadherin | | | | | ++ |
| N-cadherin | | | | | ++ |
| vimentin | +(UCD) | +++(UCD) | (+/−)(UCD) | +++ | ++ |
| p21 | | | | | |
| p27 | | | | | |
| p53 | | | | mutant | mutant |
| p63 | | | | + | |
| Snail | | | | overexpressed | |
| Akt-1 | | | | + | |
| Akt-2 | | | | — | |
| Akt-3 | | | | + | |
| Akt-pSer473 | | | | +++ | |

By fluorescence in situ hybridization (FISH) with probes for the HER2/neu region of chromosome 17 (17q1,2q12) and the centromere of chromosome 17 (D17Z1), HER2/neu is not amplified in DKAT cells. The breast and chest wall tumors were negative for HER2/neu overexpression by immunostaining (Dako®). Collectively, protein expression and immunohistochemical studies showed that the DKAT cell line is ER/PR-negative, HER2/neu is not overexpressed, EGFR is over expressed, basal-type markers are expressed (CK5/6+, CK 14+, CK 17+, CK8/18−/−, p63+), and markers of EMT are expressed (Cardiff R. D. Clin Cancer Res. 11:8534-7 (2005)). This is consistent with the recent observation that EMT markers are expressed in triple-negative breast cancer, and that triple-negative breast cancer may be prone to EMT/MET (17).

p53 sequencing: Sequencing of exons 5-9 of the TP53 gene in DKAT cells identified a single point mutation in exon 8 at codon 273 (CGT>CAT). This mutation is present in both the sense and antisense sequences, and was confirmed by repeat sequencing of a second, independent genomic DNA preparation. This is a missense mutation previously reported in the MDA-MB-468 breast cancer cell line, and reported to be deleterious to function (Debies et al., J. Clin. Invest. 118(1):51-63 (2008).
Differential gene expression: The cluster is highly similar to the Charafe-Jauffret et al. cluster (Oncogene 25(15):2273-84 (2006)). Both MDA-MB-231 and HMECs served as controls, showing high correlation to the published cell lines (r=0.70 and r=0.84, respectively), validating the effectiveness of combining these two data sets. As reported, the cell lines clearly segregate into two classes, Type I and Type II. The DKAT cells associate with basal-like cell lines, most closely with SUM-149 cells, and also with 184B5, MCF-10A, and HMEC cell lines.
Comparative pathology (Human vs. Mouse), evidence for EMT in vivo: The primary surgical specimen had eight widely separated, small multi-focal tumor lesions. Each lesion has relatively unique morphological and immunohistochemical features. However, all foci were composed of high-grade, undifferentiated cells. The tumor cells in one focus exhibited strong staining for vimentin and CK 5 and 6. This was noteworthy because the recurrent tumor on the chest wall was strongly positive for vimentin and CK 6. The IHC stain for E-cadherin was strong but was not limited to the membranes. The bone marrow had multiple metastases that were strongly positive for E-cadherin and keratin but not for vimentin.

Transplantation of DKAT cells into nude and SCID mice resulted in very slow growth patterns. Some tumors grew as invasive masses that spread through the mammary fat pad as contiguous cords of neoplastic cells. One transplant into an intact fat pad resulted in an entirely different pattern with ductal and intraductal spread through the host mouse mammary gland. These cells exhibited a much different cytological pattern with large cells with abundant cytoplasm, well demarcated cell membranes and large pleomorphic nuclei. The immunohistochemical pattern was largely consistent with malignant epithelium but scattered vimentin and smooth muscle actin positive cells were observed.

Stein Cell Marker Expression: Triple-negative breast cancers have been reported to have a higher percentage of CD44⁺/CD24^{-/low} than other breast cancer subtypes. Since the DKAT cell line was isolated from an aggressive triple-negative breast cancer, expression of the CD44 and CD24 surface markers was tested. In agreement with other reports, the triple-negative DKAT cell line was found to contain a population of CD44⁺/CD24^{-/low} cells. The percentage of cells expressing this combination of markers increases throughout passaging in culture to 50% by passage 16, after which point it remains stable.
Epithelial to Mesenchymal Transition: Since the original breast tumor from which the DKAT cells are derived exhibited morphologic evidence of EMT, the ability of DKAT cells to undergo EMT in vitro was tested. The DKAT cells are normally cultured in mammary epithelial growth medium without serum (MEGM). DKAT cells cultured in MEGM exhibit a cobblestone-like appearance and grow in tightly packed clusters. At the protein level, DKAT cells grown in MEGM express the epithelial marker, E-cadherin, and low levels of the mesenchymal marker and intermediate filament protein, vimentin.

The DKAT line was grown in stromal cell growth mediium containing 5% serum (SCGM). After 24 hrs in SCGM, DKAT cells acquired a mesenchymal appearance with many cells spreading out and others taking on a spindle-shape. Western blotting demonstrated a 95% decrease in E-cadherin expression and 100% increase in vimentin expression relative to passage-matched DKAT cells grown in MEGM. Immunofluoresence revealed that among cells cultured in SCGM there were two populations of DKAT cells. Some retained the original epithelial phenotype as evidenced by positive staining for E-cadherin alone, while the second population of cells stained positively for vimentin but not E-cadherin, indicating a switch to the mesenchymal phenotype.

Mouse Xenograft: The DKAT cell line is tumorigenic in mouse xenograft models. When injected into the mammary fat pad of NOD/SCID mice, DKAT cells consistently form tumors at doses as low as 1,000 injected cells. FACS sorted CD44+/CD24−/low cells were able to form tumors in the mammary fat pad at doses as low as 10 cells (4/10 mice). These tumors are invasive into the mammary fat pad.

Further, when DKAT cells were injected into the carotid artery of immunocompromised mice, the mice developed central nervous system metastasis to the brain parenchyma (FIGS. 2A-C) and to the leptomenges (FIG. 2D and FIG. 2E). In addition, these mice also developed lung metastasis (FIG. 2F). These data provide evidence for the DKAT cell line as a model for central nervous system metastasis as well as the novel observation that aggressive breast cancers might metastasize from the central nervous system to the lung.

Thus, the present invention provides a breast cancer cell line from tumor cells in the malignant effusion of a breast cancer patient. Although many human breast cancer cell lines have been developed from pleural fluid samples, DKAT cells are notable for the exceptional ease with which these cells adapted to in vitro growth. Given the swift acclimation to tissue culture and the presence of few if any contaminating non-epithelial cells, DKAT cells did not appear to undergo a rigorous selection process for survival in vitro. DKAT cells may therefore retain many features of the original tumor cells, including the highly invasive behavior that contributed to the rapid demise of the patient from uncontrollable and widespread breast cancer metastases.

Positive immunostaining for cytokeratin attests to the epithelial origin of the cells, and karyotype analysis confirms the human origin of the cell line. The difference in progesterone receptor (PR) status between the PR+ DKAT cells and PR− primary and chest wall tumors may be related to tumor heterogeneity as well as specific factors associated with tissue culture or antibody and tissue processing.

The epithelial mesenchymal transition/mesenchymal-epithelial transition (EMT/MET) is associated with increased invasion and metastasis [9]. Indeed, the spindle-shaped, ER negative, vimentin positive DKAT cells are similar in appearance to those from breast cancer lines such as MDA-MB-231, which exhibit invasive, metastatic behavior in vitro and in vivo. Matrigel invasion assays identify DKAT cells as highly motile and invasive. Thus, DKAT offers a useful model for studies aimed at elucidating the mechanisms underlying rapidly progressive, invasive breast cancer.

A deposit of the DKAT cell line has been made with the American Type Culture Collection (ATCC), Manassas, Va. 20110 USA on Sep. 9, 2009. The deposit has been assigned ATCC Accession Number PTA-10322. This deposit of the DKAT cell line will be maintained in the ATCC depository, which is a public depository, for a period of 30 years, or 5 years after the most recent request, or for the effective life of the patent, whichever is longer, and will be replaced if it becomes nonviable during that period. Applicants do not waive any infringement of their rights granted under this patent.

The foregoing is illustrative of the present invention, and is not to be construed as limiting thereof. The invention is defined by the following claims, with equivalents of the claims to be included therein.

All publications, patent applications, patents, patent publications, GenBank® database sequences and other references cited herein are incorporated by reference in their entireties for the teachings relevant to the sentence and/or paragraph in which the reference is presented.

### REFERENCES

1. Tavassoli F, Devilee P, editors. (2003) WHO Classification of Tumors. Pathology & Genetics: Tumors of the breast and female genital organs. Lyon (France): IARC Pres.
2. Perou et al. (2000) Molecular portraits of human breast tumours. Nature 406: 747-52.
3. Sorlie et al. (2001) Gene expression patterns of breast carcinomas distinguish tumor subclasses with clinical implications. Proc Natl Acad Sci USA 98:10869-74.
4. Sorlie et al. (2003) Repeated observation of breast tumor subtypes in independent gene expression data sets. Proc Natl Acad Sci U S A 100:8418-23.
5. Abd El-Rehim et al. (2005) High-through-put protein expression analysis using tissue microarray technology of a large well-characterised series identifies biologically distinct classes of breast cancer confirming recent cDNA expression analyses. Int J Cancer 116:340-50.
6. Nielsen et al. (2004) Immunohistochemical and clinical characterization of the basal-like subtype of invasive breast carcinoma. Clin Cancer Res 10:5367-74.
7. Rodriguez-Pinilla et al. (2006) Prognostic significance of basal-like phenotype and fascin expression in node-negative invasive breast carcinomas. Clin Cancer Res 12:1533-9.
8. Christiansen et al. (2006) Reassessing epithelial to mesenchymal transition as a prerequisite for carcinoma invasion and metastasis. Cancer Res 66:8319-26.
9. Friedl et al. (2003) Tumour-cell invasion and migration: diversity and escape mechanisms. Nat Rev Cancer 3:362-74.
10. Gupta et al. (2006) Cancer metastasis: building a framework. Cell 127:679-95.
11. Savagner P. (2001) Leaving the neighborhood: molecular mechanisms involved during epithelial-mesenchymal transition. Bioessays 23:912-23.
12. Thiery J P. (2002) Epithelial-mesenchymal transitions in tumour progression. Nat Rev Cancer 2:442-54.
13. Thompson et al. (2005) Carcinoma invasion and metastasis: a role for epithelial-mesenchymal transition? Cancer Res 65:5991-5; discussion 5.
14. Thiery et al. (2006) Complex networks orchestrate epithelial-mesenchymal transitions. Nat Rev Mol Cell Biol 7:131-42.
15. Hazan et al. (2004) Cadherin switch in tumor progression. Ann N Y Acad Sci 1014:155-63.
16. Maeda et al. (2005) Cadherin switching: essential for behavioral but not morphological changes during an epithelium-to-mesenchyme transition. J Cell Sci 118:873-87.
17. Cano et al. (2000) The transcription factor snail controls epithelial-mesenchymal transitions by repressing E-cadherin expression. Nat Cell Biol 2:76-83.
18. Bolos et al. (2003) The transcription factor Slug represses E-cadherin expression and induces epithelial to mesenchymal transitions: a comparison with Snail and E47 repressors. J Cell Sci 116:499-511.
19. Comijn et al. (2001) The two-handed E box binding zinc finger protein SIP1 downregulates E-cadherin and induces invasion. Mol Cell 7:1267-78.
20. Eger et al. (2005) DEF1 is a transcriptional repressor of E-cadherin and regulates epithelial plasticity in breast cancer cells. Oncogene 24:2375-85.
21. Perez-Moreno et al. (2001) A new role for E12/E47 in the repression of E-cadherin expression and epithelial-mesenchymal transitions. J Biol Chem 276:27424-31.
22. Yang et al. (2004) Twist, a master regulator of morphogenesis, plays an essential role in tumor metastasis. Cell 117:927-39.
23. Peinado et al. (2007) Snail, Zeb and bHLH factors in tumour progression: an alliance against the epithelial phenotype? Nat Rev Cancer 7:415-28.
24. Peinado et al. (2004) Transcriptional regulation of cadherins during development and carcinogenesis. Int J Dev Biol 48:365-75.
25. De Craene et al. (2005) The transcription factor snail induces tumor cell invasion through modulation of the epithelial cell differentiation program. Cancer Res 65:6237-44.
26. Moreno-Bueno et al. (2006) Genetic profiling of epithelial cells expressing E-cadherin repressors reveals a distinct role for snail, slug, and e47 factors in epithelial-mesenchymal transition. Cancer Res 66:9543-56.
27. Sarrio et al. (2008) Epithelial-mesenchymal transition in breast cancer relates to the basal-like phenotype. Cancer Res 68:990-7.
28. Cardiff R D. (2005) Epithelial to Mesenchymal Transition Tumors: Fallacious or Snail's Pace? Clin Cancer Res 11:8534-7.
29. Tarin et al. (2005) The fallacy of epithelial mesenchymal transition in neoplasia. Cancer Res 65:5996-6000.
30. Thompson et al. (2005) Carcinoma invasion and metastasis: a role for epithelial-mesenchymal transition? Cancer Res 65:5991-5.
31. Peinado et al. (2007) Zeb and bHLH factors in tumour progression: an alliance against the epithelial phenotype? Nat Rev Cancer 7:415-28.
32. Al-Hajj et al. (2003). Prospective identification of tumorigenic breast cancer cells. Proc. Natl. Acad. Sci. USA 100:3983-3988.
33. Reya et al. (2001). Stem cells, cancer, and cancer stem cells. Nature 414:105-11
34. Hennessy B T et al. (2009) Characterization of naturally occurring breast cancer subset enriched in epithelial to mesenchymal transition and stem cell characteristics. Cancer Res. 69: 4116-4124.
35. Mani et al. (2008). The epithelial-mesenchymal transition generates cells with properties of stem cells. Cell 133:704-715.
36. Damonte et al. (2008) Mammary carcinoma behavior is programmed in the precancer stem cell. Breast Cancer Research, (doi:10.1186/bcr2104).
37. Debies et al. (2008) Tumor escape in a Wntl-dependent mousebreast cancer model is enabled by p19Arf/p53pathway lesions but not p16Ink4a loss. J. Clin. Invest. 118(1):51-63.
38. Kreike et al. (2007) Gene expression profiling and histopathological characterization of triple-negative/basal-like breast cancers. Breast Cancer Res 9:R65.
39. Emad et al. (2007) Are Triple-negative tumors and basal-like breast cancers synonymous? Breast Cancer Res. 9:204.
40. Korkaya et al. (June 2009) Regulation of Mammary Stein/Progenitor Cells by PTEN/Akt/beta-Catenin Signaling. PLoS Biology 7 (6):e1000121.
41. Abbey et al. In vitro positron-emission tomography imaging of progression and transformation in a mouse model of mammary neoplasia. PNAS 101:11438-43 (2004).
42. Modrich, P. Mechanism in eukaryotic mismatch repair. (2006) J Biol Chem, 281, 30305-30309.
43. Lochter et al. (1997) Misregulation of stromalysin-1 expression in mouse mammary tumor cells accompanies acquisition of stromalysin-1 dependent invasive properties. J. Biol. Chem. 272:5007-5015.

That which is claimed is:

1. A cell line deposited under ATCC Accession No. PTA-10322.

2. A human triple-negative breast cancer cell line, wherein the cell line comprises the following marker profile:
high expression of Snail-1 and Snail-2 (Slug); and
a p53 mutation in exon 8 at codon 273 (CGT>CAT).

3. The cell line of claim 2, wherein the cell line is the cell line deposited under ATCC Accession No. PTA-10322.

4. A human triple-negative breast carcinoma cell line, wherein a cell of said cell line produces a solid carcinoma upon subcutaneous implantation or injection into a non-human mammal, said cell line comprising the following marker profile:
high expression of Snail-1 and Snail-2 (Slug); and
a p53 mutation in exon 8 at codon 273 (CGT>CAT).

5. The cell line of claim 4, wherein the cell line is the cell line deposited under ATCC Accession No. PTA-10322.

6. The cell line of claim 4, wherein the non-human mammal is immune deficient.

7. The cell line of claim 4, wherein the non-human mammal is a mouse.

8. A solid tumor produced in a non-human mammal, wherein said tumor is produced by introducing into said mammal a cell of the human triple-negative breast cancer cell line of claim 2.

9. The solid tumor of claim 8, wherein the non-human mammal has T-cell immunosuppression.

10. The solid tumor of claim 8, wherein the human triple-negative breast cancer cell line is the cell line deposited under ATCC Accession No. PTA-10322.

11. The solid tumor of claim 8, wherein the non-human mammal is immune deficient.

12. The solid tumor of claim 8, wherein the non-human mammal is a mouse.

13. A non-human mammal comprising one or more cells of a human triple-negative breast cancer cell line of claim 2.

14. The non-human mammal of claim 13, wherein the cell line is the cell line deposited under ATCC Accession No. PTA-10322.

15. The non-human mammal of claim 13, wherein the non-human mammal is immune deficient.

16. The non-human mammal of claim 13, wherein the non-human mammal is a mouse.

17. A method of producing a solid tumor in a non-human mammal, comprising:
introducing into said non-human mammal a cell of the human triple-negative breast cancer cell line of claim 2, wherein said cell produces a solid tumor in said mammal.

18. A method for establishing a cell line derived from a human triple-negative breast cancer tumor, comprising
a) introducing into a non-human mammal a cell of the human triple-negative breast cancer cell line of claim 2,
wherein said cell produces a solid tumor in said non-human mammal;
b) removing said tumor from said non-human mammal; and
c) culturing cells of the tumor in a culture medium, thereby establishing a cell line derived from the human triple-negative breast cancer tumor.

19. A method of identifying modulation of gene and/or protein expression and/or activity during breast cancer invasion, comprising:
a) introducing into a non-human mammal a cell of the human triple-negative breast cancer cell line of claim 2,
wherein said cell produces a solid tumor in said non-human mammal; and
b) identifying modulation of gene and/or protein expression and/or activity during breast cancer invasion resulting from the presence of said tumor.

20. The method of claim 19, where the modulation of gene and/or protein expression and/or activity comprises modulation of gene copy number, miRNA levels and type, mRNA levels, protein levels, protein phosphorylation, and any combination thereof.

21. A method of identifying modulation of gene expression during metastasis, comprising:
a) introducing into a non-human mammal a cell of the human triple-negative breast cancer cell line of claim 2, wherein said cell produces a solid tumor in said non-human mammal; and
b) identifying modulation in gene expression during metastasis resulting from the presence of said tumor.

22. A method of identifying modulation in gene expression during epithelial to mesenchymal transition and mesenchymal to epithelial transition comprising:
a) introducing into a non-human mammal a cell of the human triple-negative breast cancer cell line of claim 2
wherein said cell produces a solid tumor in said non-human mammal; and
b) identifying modulation in gene expression during epithelial to mesenchymal transition and mesenchymal to epithelial transition resulting from the presence of said tumor.

23. A method of identifying a chemotherapeutic agent having a therapeutic effect on a triple-negative breast cancer cell, comprising:
a) contacting a human triple-negative breast cancer cell from the cell line of claim 2 with a chemotherapeutic agent; and
b) determining if the chemotherapeutic agent inhibits proliferation of the triple-negative breast cancer cell, whereby identification of a chemotherapeutic agent that inhibits proliferation of the triple-negative breast cancer cell identifies a chemotherapeutic agent having a therapeutic effect on a triple-negative breast cancer cell.

24. A method of identifying a chemotherapeutic agent having a therapeutic effect on triple-negative breast cancer, comprising:
a) introducing into a non-human mammal a cell of the human triple-negative breast cancer cell line of claim 2,
wherein said cell produces a solid tumor in said non-human mammal;
b) administering a chemotherapeutic agent to the non-human mammal with said solid tumor; and
c) determining if the chemotherapeutic agent inhibits proliferation of the tumor, whereby identification of a chemotherapeutic agent that inhibits proliferation of the tumor identifies a chemotherapeutic agent having a therapeutic effect on triple-negative breast cancer.

25. A method of identifying a biological agent having a therapeutic effect on a triple-negative breast cancer cell, comprising
a) contacting a human triple-negative breast cancer cell from the cell line of claim 2 with a biological agent; and
b) determining if the biological agent inhibits the proliferation of the triple-negative breast cancer cell, whereby identification of a biological agent that inhibits proliferation of the triple-negative breast cancer cell identifies a biological agent having a therapeutic effect on a triple-negative breast cancer cell.

26. A method of identifying a biological agent having a therapeutic effect on triple-negative breast cancer, comprising
a) introducing into a non-human mammal a cell of the human triple-negative breast cancer cell line of claim 2,
wherein said cell produces a solid tumor in said non-human mammal;
b) administering a biological agent to the non-human mammal with said solid tumor; and
c) determining if the biological agent inhibits proliferation of the solid tumor, whereby identification of a biological agent that inhibits tumor proliferation identifies a biological agent having a therapeutic effect on triple-negative breast cancer.

27. A method of screening for modulators of gene expression, comprising:
a) measuring the level of gene expression in a cell from the cell line of claim 2;
b) contacting said cell with a candidate agent;
c) measuring the level of gene expression in said cell after contact with the candidate agent,
whereby a difference in the level of gene expression after contact with the candidate agent as compared to before contact with the candidate agent identifies a modulator of gene expression.
